SEQUENCE LISTING (12 sequences)

SEQ ID NO: 1 - protein, 16 aa, Artificial Sequence
  Synthetic Affinity Peptide
HLIHNVHKEEHAHAHN

SEQ ID NO: 2 - protein, 18 aa, Artificial Sequence
  Synthetic Affinity Peptide
HDDHDDHDDHDDHDDHDD

SEQ ID NO: 3 - protein, 18 aa, Artificial Sequence
  Synthetic Affinity Peptide
HEEHEEHEEHEEHEEHEE

SEQ ID NO: 4 - protein, 18 aa, Artificial Sequence
  Synthetic Affinity Peptide
HDEHDEHDEHDEHDEHDE

SEQ ID NO: 5 - protein, 18 aa, Artificial Sequence
  Synthetic Affinity Peptide
HEDHEDHEDHEDHEDHED

SEQ ID NO: 6 - protein, 4 aa, human
IEGR

SEQ ID NO: 7 - protein, 6 aa, human
LVPRGS

SEQ ID NO: 8 - protein, 8 aa, human
HPFHLVIH

SEQ ID NO: 9 - protein, 10 aa, human
CYPYDVPDYA

SEQ ID NO: 10 - protein, 8 aa, human
DYKDDDDK

SEQ ID NO: 11 - protein, 11 aa, human
CEQKLISEEDL

SEQ ID NO: 12 - protein, 5 aa, human
DDDDK

(19) United States
(12) Patent Application Publication
(10) Pub. No.: US 2004/0180415 A1
(43) Pub. Date: Sep. 16, 2004

(54) Methods and compositions for protein purification

(76) Inventors: Grigoriy S. Tchaga, Newark, CA (US); George G. Jokhadze, Mountain View, CA (US)

Correspondence Address: BOZICEVIC, FIELD & FRANCIS (BD BIOSCIENCES), 200 MIDDLEFIELD ROAD, SUITE 200, MENLO PARK, CA, 94025, US

(21) Appl. No.: 10/762,588

(22) Filed: Jan. 21, 2004

Related U.S. Application Data
(63) Continuation-in-part of application No. 09/858,332, filed on May 15, 2001.
(60) Provisional application No. 60/441,804, filed on Jan. 21, 2003.

(51) Int. Cl. C12N 9/00

(52) U.S. Cl. 435/183; 530/412

(57) ABSTRACT

The present invention provides methods of purifying proteins that include a metal ion affinity peptide. The methods generally involve contacting a fusion protein that includes a metal ion affinity peptide with at least two different metal ion chelating resins. In certain representative embodiments, the methods include contacting a fusion protein with a first metal ion chelate resin having a first immobilized metal ion; eluting any bound protein from the first metal ion chelate resin, to produce an eluate; contacting the eluate with a second metal ion chelate resin having a second immobilized metal ion; and eluting any bound protein from the second metal ion chelate resin. Also provided are kits for use in practicing the subject methods. The subject methods find use in a variety of protein purification applications.

### CROSS-REFERENCE TO RELATED APPLICATIONS

This application is a continuation-in-part of U.S. patent application Ser. No. 09/858,332, filed May 15, 2001, which application is incorporated herein by reference in its entirety.

This application also claims the benefit of U.S. Provisional Patent Application No. 60/441,804, filed Jan. 21, 2003; which application is incorporated herein by reference in its entirety.

### FIELD OF THE INVENTION

This invention relates generally to the field of protein chemistry. Specifically, the present invention relates to the field of protein purification methods based on metal ion affinity site compositions.

### BACKGROUND OF THE INVENTION

Immobilized Metal Ion Affinity Chromatography (IMAC) is one of the most frequently used techniques for purification of fusion proteins containing affinity sites for metal ions. IMAC is a separation principle that utilizes the differential affinity of proteins for immobilized metal ions to effect their separation. This differential affinity derives from the coordination bonds formed between metal ions and certain amino acid side chains exposed on the surface of the protein molecules. Since the interaction between the immobilized metal ions and the side chains of amino acids has a readily reversible character, it can be utilized for adsorption and then be disrupted using mild (i.e., non denaturing) conditions.

Adsorbents that are currently commercially available include iminodiacetic acid (IDA), nitriloacetic acid (NTA), caboxymethylated aspartic acid (CM-Asp), and tris-carboxymethyl ethylene diamine (TED). These ligands offer a maximum of tri-(IDA), tetra-(NTA, CM-Asp), and penta-dentate (TED) complexes with the respective metal ion. In most commercially available adsorbents, metal chelating ligands are provided at an average density of about 12Δ. Depending on the ligand, various metals can be chelated. Metal ions typically used in IMAC procedures have been classified into three categories—hard, intermediate, and soft—based on their preferential reactivity toward nucleophiles. The hard metal ions Fe³⁺, Ca²⁺, and Al³⁺ show a preference for oxygen; the soft metal ions Cu⁺, Hg²⁺, Ag⁺, and the like show a preference for sulfur; and intermediate metal ions such as Cu²⁺, Ni²⁺, Zn²⁺, and Co²⁺ coordinate nitrogen, oxygen, and sulfur. The number of cysteine residues on the surfaces of proteins is limited; therefore, histidine residues are the major targets for intermediate metal ions.

The observation that histidine residues bind to certain immobilized ions led to the development of histidine-containing “tags” for proteins to aid in purification of such proteins. In particular, peptide tags containing multiple histidines have been developed.

For example, hexa-histidine tags are commonly used with IMAC adsorbents for purification of recombinant proteins.

Despite the advances made in protein purification using IMAC, there is an ongoing need in the art for improved metal ion affinity tags for use in purifying proteins. The present invention addresses this need.

### Literature

The following publications are of interest: Itakura, et al., Science 198:1056-63 (1977); Germino, et al., Proc. Natl. Acad. Sci. U.S.A. 80:6848-52 (1983); Nilsson et al., Nucleic Acids Res. 13:1151-62 (1985); Smith et al., Gene 32:321-27 (1984); Dobeli, et al., U.S. Pat. No. 5,284,933; Dobeli, et al., U.S. Pat. No. 5,310,663; U.S. Pat. No. 4,569,794; and U.S. Pat. No. 5,594,115.

### SUMMARY OF THE INVENTION

The present invention provides methods of purifying proteins that include a metal ion affinity peptide. The methods generally involve contacting a fusion protein that includes a metal ion affinity peptide with at least two different metal ion chelating resins. In many embodiments, the methods including contacting a fusion protein with a first metal ion chelate resin having a first immobilized metal ion; eluting any bound protein from the first metal ion chelate resin, to produce an eluate; contacting the eluate with a second metal ion chelate resin having a second immobilized metal ion; and eluting any bound protein from the second metal ion chelate resin. Also provided are kits for use in practicing the subject methods. The subject methods find use in a variety of protein purification applications.

### BRIEF DESCRIPTIONS OF THE DRAWINGS

FIG. 1 depicts an exemplary protein purification scheme.

FIG. 2 depicts gel electrophoresis analysis of various fractions from the purification scheme described in Example 1 and shown in FIG. 1.

### DEFINITIONS

The terms “affinity peptide,” “high affinity peptide,” and “metal ion affinity peptide” are used interchangeably herein to refer to a histidine-rich peptide that binds to a metal ion.

The terms “protein of interest” and “fusion partner polypeptide,” used interchangeably herein, refer to any protein to which the affinity peptide is fused for the purpose of purification or immobilization.

As used herein, the term “fusion protein” refers to the protein hybrid comprising a metal ion affinity peptide and a fusion partner polypeptide.

As used herein, the terms “secretion sequence” or “secretion signal sequence” refer to an amino acid signal sequence which leads to the transport of a protein containing the signal sequence outside the cell membrane. In the present case, a fusion protein of the present invention may contain such a secretion sequence to enhance and simplify purification.

As used herein, the term “proteolytic cleavage site” refers to any amino acid sequence recognized by any proteolytic enzyme. In the present case, a fusion protein of the present invention may contain such a proteolytic cleavage site between the protein of interest and the affinity peptide and/or other amino acid sequences so that the protein of interest may be separated easily from these heterologous amino acid sequences.

As used herein, the term “enterokinase” refers to a protease which cleaves peptide chains specifically at the primary amino acid sequence: Asp-Asp-Asp-Asp-Lys (SEQ ID NO:12).

As used herein, the terms “recombinant proteolytic enzyme”, “recombinant protease”, “engineered proteolytic enzyme” or “engineered protease” refer to proteolytic enzymes or proteases that contain a histidine-rich affinity peptide.

As used herein, the term “metal ion” refers to any metal ion for which the affinity peptide has affinity and that can be used for purification or immobilization of a fusion protein. Such metal ions include, but are not limited to, Ni⁺², Co⁺², Fe⁺³, Al⁺³, Zn⁺² and Cu⁺². As used herein, the term “hard metal ion” refers to a metal ion that shows a binding preference for oxygen. Hard metal ions include Fe³⁺, Ca²⁺, and Al³⁺. As used herein, the term “soft metal ion” refers to a metal ion that shows a binding preference of sulfur. Soft metal ions include Cu⁺, Hg²⁺, and Ag⁺. As used herein, the term “intermediate metal ion” refers to a metal ion that coordinates nitrogen, oxygen, and sulfur. Intermediate metal ions include Cu²⁺, Ni²⁺, Zn²⁺, and Co²⁺.

As used herein, the terms “adsorbent” or “solid support” refer to a chromatography or immobilization medium used to immobilize a metal ion.

As used herein, the term “regeneration,” in the context of the fusion protein, refers to the process of separating or eliminating the affinity peptide and other heterologous amino acid sequences from the fusion protein to render the protein of interest after purification in its wild-type form.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glove ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells And Enzymes” (IRL Press, (1986)); B. Perbal, “A Practical Guide to Molecular Cloning” (1984).

The term “vector” refers to a replicon, such as a plasmid, a phage, a viral vector, a minichromosome, an artificial chromosome, or a cosmid, to which another DNA segment may be attached so as to bring about the replication of the attached segment.

The terms “DNA molecule,” “polynucleotide,” and “nucleic acid molecule” are used interchangeably herein and refer to the polymeric form of deoxyribonucleotides (adenine, guanine, thymine, or cytosine) in either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA). The terms refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

A DNA “coding sequence” is a double-stranded DNA sequence which is transcribed and translated into a polypeptide in vivo (e.g., in a living cell) or in vitro (e.g., in a cell-free system) when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. A polyadenylation sequence may also be located 3′ to the coding sequence.

The terms “polypeptide” and “protein”, used interchangebly herein, refer to a polymeric form of amino acids of any length, which can include coded and non-coded amino acids, chemically or biochemically modified or derivatized amino acids, and polypeptides having modified peptide backbones. Polypeptides may be polymers of: (a) naturally occurring amino acid residues; (b) non-naturally occurring amino acid residues, e.g. N-substituted glycines, amino acid substitutes, etc.; or (c) both naturally occurring and non-naturally occurring amino acid residues/substitutes. This term does not refer to or exclude post-translational modifications of the polypeptide, for example, glycosylations, acetylations, phosphorylations and the like. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein the term “isolated polypeptide” is meant to describe a polypeptide that is in an environment different from that in which the polypeptide naturally occurs. As used herein, the term “substantially purified polypeptide” refers to a polypeptide that is removed from its natural environment and is at least 60% free, at least 75% free, or at least 90% free from other components with which it is naturally associated. The term “substantially purified polypeptide” also refers to a polypeptide that is at least about 60% free, at least about 70% free, at least about 75% free, at least about 80% free, at least about 85% free, at least about 90% free, at least about 95% free, at least about 98% free, or at least about 99% free, of macromolecules other than the polypeptide found in a sample comprising the polypeptide before the polypeptide is purified.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defiling the present invention, the promoter sequence is bounded at it 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “selection gene” refers to a gene that enables the discrimination of cells displaying a required phenotype upon implementation of certain conditions. For example, the growth of bacteria in medium containing antibiotics to select for the bacterial cells containing antibiotic resistance genes.

The term “oligonucleotide” or “probe” as used herein, refers to a molecule comprised of ribonucleotides or deoxyribonucleotides. The exact size of the oligonucleotide or probe will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, the source of primer and the method used.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence or hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A cell line” is a clone of a primary cell that is capable of stable growth in vitro for many generations.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a “recombinant host cell.” Host cells include eukaryotic and prokaryotic cells.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art.

A “heterologous” region of the DNA construct is an identifiable segment of DNA within a larger DNA molecule that is not found in association with the larger molecule in nature. Thus, when the heterologous region is a mammalian gene, the gene will usually be flanked by DNA that does not flank the mammalian genomic DNA in the genome of the source organism. In another example, a heterologous region is a coding sequence where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The amino acids described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxyl group present at the carboxyl terminus of a polypeptide. Abbreviations for amino acid residues are (in the following order: one-letter symbol, three-letter symbol, amino acid): Y, Tyr, tyrosine; G, Gly, glycine; F, Phe, phenylalanine; M, Met, methionine; A, Ala, alanine; S, Ser, serine; I, Ile, isoleucine; L, Leu, leucine; T, Thr, threonine; V, Val, valine; P, Pro, proline; K, Lys, lysine; H, His, histidine; Q, Gln, glutamine; E, Glu, glutamic acid; W, Trp, tryptophan; R, Arg, arginine; D, Asp, aspartic acid; N, Asn, asparagine; C, Cys, cysteine. It should be noted that all amino acid residue sequences are represented herein by formulae whose left and right orientation is in the conventional direction of amino terminus to carboxyl terminus. Furthermore, it should be noted that a dash at the beginning or end of an amino acid residue sequence indicates a peptide bond or non-standard peptide linkage to a further sequence of one or more amino acid residues.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a metal ion affinity peptide” includes a plurality of such peptides and reference to “the purification method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The invention provides methods for purification of proteins that include a metal ion affinity peptide, and kits for carrying out a subject method. The subject invention finds use in a variety of protein purification applications.

Fusion proteins comprising a metal ion affinity peptide are purified using immobilized metal ion affinity chromatography (IMAC). In some embodiments, a metal ion affinity peptide has affinity to both hard and intermediate metal ions. Thus, two IMAC resins, each having immobilized thereon a different metal ion, e.g., a hard and an intermediate metal ion, can be used with a single metal ion affinity peptide. Use of two different metal ions for purification of a protein tagged with a single metal ion affinity peptide is advantageous, as a high degree of purification can be attained with a single chromatographic step.

### METHODS

The present invention provides methods for purifying proteins that include a metal ion affinity peptide (e.g., fusion proteins that include a metal ion affinity peptide and a fusion partner). The methods generally involve contacting a fusion protein that includes a metal ion affinity peptide with at least two different metal ion chelating resins. In many embodiments, the methods include contacting the fusion protein with a first metal ion chelate resin having a first immobilized metal ion; eluting any bound protein from the first metal ion chelate resin, to produce an eluate; contacting the eluate with a second metal ion chelate resin having a second immobilized metal ion; and eluting any bound protein from the second metal ion chelate resin. One or more washing steps may optionally be included to remove undesired components of the sample applied to the resin. Two or more different resins may be used.

In general, a fusion protein that includes a metal ion affinity peptide is synthesized (e.g., by a recombinant host cell); and the fusion protein is purified using a metal ion chelate resin. The subject methods provide for purification of a fusion protein from a sample which contains, in addition to a fusion protein, other components e.g., proteins other than a fusion protein, and other non-protein components such as non-protein macromolecules. The starting sample is any sample containing a fusion protein and one or more other components. Using a method of the invention, a fusion protein can be purified in one, two, or more chromatographic steps.

In some embodiments, a fusion protein is purified in one chromatographic step. A single chromatographic step includes contacting a sample with an IMAC resin such that a fusion protein contained within the sample binds to the IMAC resin, and eluting the bound fusion protein.

In other embodiments, a fusion protein is purified in two chromatographic steps.

For example, a sample that includes a fusion protein is contacted with a first IMAC resin having a first immobilized metal ion, under conditions that favor binding of the fusion protein to the first immobilized metal ion. The fusion protein is eluted from the first IMAC resin, then contacted with a second IMAC resin having a second immobilized metal ion, under conditions that favor binding of the fusion protein to the second immobilized metal ion. The fusion protein is then eluted from the second IMAC resin.

Using a method as described herein, a fusion protein is purified to a desired degree, depending on the application. In some embodiments, a fusion protein purified using a subject method is at least about 60%, at least about 70%, at least about 80%, at least about 85%, at least about 90%, at least about 95%, at least about 98%, at least about 99%, or more, pure, e.g., free of macromolecules other than the polypeptide found in a sample comprising the polypeptide before the polypeptide is purified. Purity can be determined using any known method, including, but not limited to, sodium dodecyl sulfate-polyacrylamide gel electrophoresis (SDS-PAGE) separation following by staining (e.g., Coomassie blue, silver staining, etc.).

The recovery of the fusion protein (e.g., the yield) is at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, at least about 98%, or more.

### Use of Two Different Metal Ions

As discussed above, in some embodiments, a subject purification method involves use of two different metal ions. In some of these embodiments, a subject method for purifying a fusion protein involves the following steps: a) contacting a sample that contains a fusion protein having a metal ion affinity peptide with a first metal ion chelate resin having a first metal ion immobilized on the first metal ion chelate resin, where the contacting step is typically carried out under conditions that favor binding of the fusion protein to the first immobilized metal ion; b) eluting any bound fusion protein from the first metal ion chelate resin, to produce a first eluate; c) contacting the first eluate with a second metal ion affinity resin having a second metal ion immobilized on the second metal ion chelate resin, where the contacting step is typically carried out under conditions that favor binding of the fusion protein to the second immobilized metal ion; and d) eluting any bound fusion protein from the second metal ion chelate resin, to produce a second eluate that includes the purified fusion protein. In these embodiments, as discussed above, one or more washing steps are included between steps (a) and (b) and/or between steps (c) and (d), to remove unadsorbed material and/or to remove other, undesired, macromolecules.

In some embodiments, the first metal ion chelate resin having a first metal ion immobilized on the first metal ion chelate resin, and the second metal ion chelate resin having a second metal ion immobilized on the second metal ion chelate resin are contained within one purification module, e.g., are contained within one column. In other embodiments, the first metal ion chelate resin having a first metal ion immobilized on the first metal ion chelate resin, and the second metal ion chelate resin having a second metal ion immobilized on the second metal ion chelate resin are contained within two separate purification modules, e.g., are contained within two separate columns.

In some of these embodiments, the first metal ion chelate resin and the second metal chelate resin are the same. In other embodiments, the first metal ion chelate resin and the second metal ion chelate resin are different. In some embodiments, the first immobilized metal ion is a hard metal ion (e.g., Fe³⁺, Ca²⁺, or Al³⁺); and the second immobilized metal ion is an intermediate metal ion (e.g., Cu²⁺, Ni²⁺, Zn²⁺, Co²⁺). In other embodiments, the first immobilized metal ion is an intermediate metal ion (e.g., Cu²⁺, Ni²⁺, Zn ²⁺, Co²⁺); and the second immobilized metal ion is a hard metal ion (e.g., Fe³⁺, Ca²⁺, or Al³⁺).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Co²⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Fe³⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Fe³⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Co²⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Ni²⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Fe³⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Fe³⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Ni²⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Zn²⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Fe³⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Fe³⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Zn²⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Cu²⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Fe³⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

In one non-limiting example, a subject method for purifying a fusion protein involves a) contacting a sample that includes a fusion protein, having a metal ion affinity peptide, with a first metal ion chelate resin having immobilized thereon a first metal ion, where the first metal ion is Fe³⁺, where the contacting is conducted under conditions that favor binding of the fusion protein to the immobilized first metal ion; b) eluting any bound fusion protein from the first metal ion chelate resin, to produce a first eluate that contains the fusion protein; c) contacting the first eluate with a second metal ion chelate resin having immobilized thereon a second metal ion, where the second metal ion is Cu²⁺, where the contacting is conducted under conditions that favor binding of the fusion protein to the immobilized second metal ion; and d) eluting any bound fusion protein from the second metal ion chelate resin, to produce a second eluate that contains the fusion protein, where the fusion protein is purified. In some embodiments, one or more washing steps are included between steps (a) and (b) and/or between steps (c) and (d).

The present invention provides any of the above-described methods, modified to substitute Fe³⁺ with Ca²⁺ or with Al³⁺.

In some embodiments, any of the above-described methods is carried out under native conditions, e.g., equilibration, wash, and elution steps are carried out under conditions such that the fusion protein substantially retains its native conformation. Suitable buffers are described in more detail below. Non-limiting examples of equilibration buffers for native protein purification include a solution containing 50 mM sodium phosphate, 0.3 M NaCl, pH 7; and a solution containing 50 mM sodium phosphate, 0.3 M NaCl, pH 8. A non-limiting example of a wash buffer for native protein purification is 50 mM sodium phosphate, 0.3 M NaCl, pH 7. A non-limiting example of an elution buffer for native protein purification is 0.15 M imidazole, pH 7. Non-limiting examples of equilibration buffers for denaturing protein purification include 50 mM sodium phosphate, 6 M guanidine-HCl, and 300 mM NaCl, pH 7.0; and 50 mM sodium phosphate, 6 M guanidine-HCl, and 300 mM NaCl, pH 8.0. A non-limiting example of a wash buffer for denaturing protein purification is 50 mM sodium phosphate, 6 M guanidine-HCl, 300 mM NaCl, pH 8.0. A non-limiting example of an elution buffer for denaturing protein purification is 45 mM sodium phosphate, 5.4 M guanidine-HCl, 270 mM NaCl, and 150 mM imidazole, pH 7.0.

Those skilled in the art will recognize that various combinations of hard and intermediate metal ion-affinity chelate resins can be used, and that the above embodiments are provided as examples of suitable purification protocols.

### Samples and Sample Preparation

Suitable samples include, but are not limited to, cell supernatants (e.g., cell culture medium), tissue extracts, crude cell lysates, cell sonicates, fermentation harvests, and the like. In some embodiments, a sample is subjected to one or more treatments before being applied to a metal ion chelate resin. In some embodiments, the fusion protein is secreted into the culture medium in which cells are grown, and the culture medium is applied directly to a metal ion chelate resin. In other embodiments, the fusion protein remains intracellular (e.g., in the cytoplasm, in a cell membrane, or in an organelle), in which case the cells are disrupted. A variety of protocols for disrupting cells to release an intracellular protein are known in the art, and can be used to extract a fusion protein from a cell. Such protocols are found in numerous publications, including, e.g., Current Protocols in Molecular Biology, (F. M. Ausubel, et al., Eds. 1987, and updates). Whether cell culture medium (“culture supernatant”) or disrupted cells (“cell lysate”) are used as the starting material, the starting material may be subjected to one or more treatments before being applied to a metal ion chelating resin. Such treatments include, but are not limited to, centrifugation, to remove cell debris, etc.; salt precipitation; application to a size exclusion chromatographic column; and application to an ion exchange chromatographic column.

### Metal Ion Affinity Resins

Any of a variety of available metal ion chelating resins can be used. In general, a metal ion chelating resin includes a carrier matrix, optionally a spacer, and a moiety that comprises a metal ion, e.g., an organic ligand that immobilizes a metal ion. Carrier matrices include, but are not limited to, cross-linked dextrans, polystyrenes, nylon, agarose, and polyacrylamides. Non-limiting examples of suitable, commercially available carrier matrices include Sepharose®6B-CL (6% cross-linked agarose; Pharmacia); Superflow™ (6% cross-linked agarose; Sterogene Bioseparations, Inc.), Uniflow™ (4% cross-linked agarose; Sterogene Bioseparations, Inc.); silica matrices; and the like. A carrier matrix is modified with a metal chelating ligand, to produce a metal chelating resin. Metal chelating ligands include, but are not limited to, carboxymethyl aspartate (CM-Asp); iminodiacetic acid (IDA); tris(carboxymethyl)ethylene diamine (TED); nitrilo triacetic acid (NTA). Several of these are commercially available. Methods of modifying a carrier matrix with a metal chelating ligand are known in the art. For example, Chaga et al. ((1999) Biotechnol. Appl. Biochem. 29:19-24) discusses modification of agarose matrix with CM-aspartate.

The metal ion chelating resin can be provided in the form of a chromatography column, e.g., wherein the resin is packed in a column. The resin can also comprise a matrix that is a solid support of any shape or configuration. Thus, the term “resin,” as used herein, refers to a resin comprising a matrix in any form, e.g., a bead, a sheet, a well, and the like. Where the resin is a bead, the beads are provided in various sizes, depending, in part, on the nature of the sample being applied, where suitable bead sizes include from about 10 μm to about 500 μm, e.g., from about 10 μm to about 20 μm, from about 16 μm to about 24 μm, from about 20 μm to about 50 μm, from about 50 μm to about 100 μm, from about 60 μm to about 160 μm, from about 100 μm to about 200 μm, from about 100 μm to about 300 μm, from about 200 μm to about 300 μm, or from about 300 μm to about 500 μm. Non-limiting examples of formats in which a metal ion chelating resin is provided include a gravity-flow column; a fast protein liquid chromatographic (FPLC) column; a multi-well (e.g., 96-well) column format; a spin column; and the like.

Metal ions metal ions can be divided into three categories (hard, intermediate and soft) based on their preferential reactivity towards nucleophiles. To the group of hard metal ions belong Fe³⁺, Ca²⁺ and Al³⁺ which show a preference for oxygen. Soft metal ions such as Cu⁺, Hg²⁺, Ag⁺, etc, prefer sulfur. Intermediate metal ions (Cu²⁺, Ni²⁺, Zn²⁺, Co²⁺) coordinate nitrogen, oxygen and sulfur. Histidine residues bind intermediate metal ions with high affinity. The binding constant of an average protein with a single histidyl residue is about 4.5×10³ M⁻¹.

In some embodiments, a metal ion chelate resin is a Co²⁺-immobilizing resin. Such resins are described in U.S. Pat. No. 5,962,641, the contents of which are incorporated herein by reference.

In some embodiments, the invention provides methods of purifying a fusion protein using multiple metal ion affinity resins, e.g., two or more different metal ion affinity resins. The multiple metal ion affinity resins can be provided in the same column, e.g., mixed together, or layered one on top of the other; or provided in two separate, tandem columns. In some embodiments, a first metal ion affinity resin comprises a matrix, a first metal ion chelating ligand, and a first immobilized metal ion, wherein the first metal ion is a hard metal ion, e.g., Fe³⁺, Ca²⁺, or Al³⁺; and a second metal ion affinity resin comprises a matrix, a second metal ion chelating ligand, and a second immobilized metal ion, wherein the second immobilized metal ion is an intermediate metal ion, e.g., Cu²⁺, Ni²⁺, Zn²⁺, Co²⁺. In other embodiments, a first metal ion affinity resin comprises a matrix, a metal ion chelating ligand, and a first immobilized metal ion, wherein the first metal ion is an intermediate metal ion, e.g., Cu²⁺, Ni²⁺, Zn²⁺, Co²⁺; and a second metal ion affinity resin comprises a matrix, a metal ion chelating ligand, and a second immobilized metal ion, wherein the second immobilized metal ion is a hard metal ion, e.g., Fe³⁺, Ca²⁺ and Al³⁺. In these embodiments, the first and second metal ion affinity resins comprise the same metal ion ligand. In some embodiments, a sample comprising a fusion protein is applied to a first resin, the resin washed to remove unbound components of the sample, bound fusion protein eluted from the first resin, and the eluted fusion protein applied to the second resin, followed by washing and eluting steps.

### Conditions for Binding

The conditions under which a protein sample comprising a fusion protein is applied to a metal ion affinity resin will vary according to various parameters, including the inherent properties of the fusion protein, the properties of the undesired components of the protein sample, etc. Generally, the sample is applied to the metal ion affinity resin, and the resin is equilibrated with a solution (e.g., an equilibration buffer). Non-limiting examples of suitable equilibration buffers include a solution containing 50 mM sodium phosphate and 0.3 M NaCl, pH 7; a solution containing 50 mM sodium phosphate and 0.3 M NaCl, pH 8; and the like. “Conditions for binding” include a condition of the sample being applied, as well as any equilibration conditions. Those skilled in the art can readily determine appropriate conditions for binding of a fusion protein in a sample to a metal ion affinity resin, based on known and determined properties of the fusion protein, etc. Conditions may be chosen such that a fusion protein retains its native conformation and/or activity. For example, a fusion protein comprising a polypeptide derived from an extreme halophile may be contacted with a metal ion affinity resin under high salt (e.g., 1.5 to 3 M NaCl). Salt concentrations suitable for applying a sample comprising a fusion protein to a metal ion affinity resin range from about 0.01 M NaCl to about 3 M NaCl, from about 0.05 M NaCl to about 1.5 M NaCl, from about 0.1 M NaCl to about 1.0 M NaCl, or from about 0.2 M NaCl to about 0.5 M NaCl. The pH conditions suitable for applying a sample comprising a fusion protein to a metal ion affinity resin range from about 3.5 to about 11, from about 4.0 to about 10.0, from about 4.5 to about 9.5, from about 5.0 to about 9.0, from about 5.5 to about 8.5, from about 6.0 to about 8.0, or from about 6.5 to about 7.5. Temperature conditions suitable for applying a sample comprising a fusion protein to a metal ion affinity resin range from about 15° C. to about 40° C., from about 20° C. to about 37° C., or from about 22° C. to about 25° C. Various additional substances may be included, including, but not limited to, detergents (e.g., sodium dodecyl sulfate, e.g., from about 0.05% to about 2%); non-ionic detergents, e.g., Tween 20™, and the like; chaotropic agents and denaturants, e.g., urea, and guanidinium HCl; buffers, e.g., Tris-based buffers, borate-based buffers, phosphate-based buffers, imidazole, HEPES, PIPES, MOPS, PIPES, TES, and the like. Non-limiting examples of a denaturing equilibration buffer include a solution containing 50 mM sodium phosphate, 6 M guanidine-HCl, and 300 mM NaCl, pH 7.0; a solution containing 50 mM sodium phosphate, 6 M guanidine-HCl, and 300 mM NaCl, pH 8.0; and the like.

### Purification Steps

In some embodiments, the invention provides a method of purifying a fusion protein from a sample comprising the fusion protein, comprising contacting a sample comprising the fusion protein with an immobilized metal ion affinity resin under conditions which favor binding of the fusion protein to the immobilized metal ion, thereby immobilizing the fusion protein; and eluting the immobilized fusion protein.

In other embodiments, the methods comprise contacting a sample comprising a fusion protein with a first immobilized metal ion affinity resin comprising a first immobilized metal ion and a second immobilized metal ion affinity resin comprising a second immobilized metal ion, wherein the fusion protein comprises a fusion partner polypeptide and a metal ion affinity peptide, and wherein the affinity peptide has a first affinity to a first immobilized metal ion and a second affinity to a second immobilized metal ion. In these embodiments, multiple resins, as described above, are used. The first affinity is generally at least about 50%, at least about 100% (or 2-fold), at least about 4-fold, at least about 5-fold, at least about 7-fold, at least about 10-fold, at least about 20-fold, at least about 50-fold, or at least about 100-fold, or more, higher than the second affinity.

In one non-limiting example, in a purification scheme utilizing two different immobilized metal ions, a sample including a fusion protein is applied to a first column containing a first resin with a first immobilized metal ion under conditions that favor binding of the fusion protein to the first immobilized metal ion. The first column is washed to remove any unbound components of the sample. The bound fusion protein is eluted, then applied to a second column containing a second resin with a second immobilized metal ion under conditions that favor binding of the fusion protein to the second immobilized metal ion. The second column is washed to remove any unbound components, and the bound fusion protein is eluted.

### Washing

One or more washing steps may be included, to remove undesired components. A washing step may be performed after a fusion protein is immobilized on a resin. The composition and temperature of a washing solution may vary according to the desired result. The optimal composition and temperature of a washing solution can readily be determined by those skilled in the art, based on known properties of the immobilized fusion protein. Wash solutions may comprise a buffer, and may further comprise additional components, as necessary, including, but not limited to, a detergent. A non-limiting example of a suitable wash buffer is a solution containing 50 mM sodium phosphate and 0.3 M NaCl, pH 7; and the like. A non-limiting of a denaturing wash buffer is a solution containing 50 mM sodium phosphate, 6 M guanidine-HCl, and 300 mM NaCl, pH 7.0.

### Eluting

The immobilized fusion protein can be eluted using a pH gradient; addition of a competitor, e.g., an organic acid, phosphates; addition of a displacer such as imidazole; and the like. A non-limiting example of an elution buffer is a solution containing 0.15 M imidazole, pH 7. A non-limiting example of a denaturing elution buffer is a solution containing 45 mM sodium phosphate, 5.4 M guanidine-HCl, 270 mM NaCl, and 150 mM imidazole, pH 7.0.

### FUSION POLYPEPTIDES

The above-described methods are useful for purifying fusion proteins that include a metal ion affinity peptide. The presence of the metal ion affinity peptide in a fusion protein allows purification of the fusion protein on a metal chelating resin.

### Metal Ion Affinity Peptides

A wide variety of metal ion affinity peptides are known in the art.

Metal ion affinity peptides are generally from about 6 to about 30, from about 7 to about 25, from about 8 to about 20, from about 9 to about 18, from about 10 to about 16, or from about 12 to about 14 amino acids in length.

In some embodiments, metal ion affinity peptides contain from about 30% to about 50%, from about 33% to about 45%, from about 35% to about 43%, or from about 37% to about 40%, histidine residues. For example, a metal ion affinity peptide 18 amino acids in length contains 6, 7, or 8 histidine residues.

In some embodiments, a metal ion affinity peptide includes 2-6 adjacent histidine residues, e.g., a metal ion affinity peptide includes (His)n, where n=2, 3, 4, 5, or 6. In some embodiments, a metal ion affinity peptide has the formula R₁-(His)ₙ-R₂, where R₁ is hydrogen, or from 1 to about 30 amino acids, where n=2-6, and where R₂ is Q, Q-Ile-Glu-Gly-Arg- or Q-Asp-Asp-Asp-Asp-Lys-, where Q is a peptide bond or from 1 to about 30 amino acids. See, e.g., U.S. Pat. No. 5,310,663.

In other embodiments, a metal ion affinity peptide has the formula His-X, where X is chosen from -Gly-His, -Tyr, -Gly, -Trp, -Val, -Leu, -Ser, -Lys, -Phe, -Met, -Ala, -Glu, -Ile, -Thr, -Asp, -Asn, -Gln, -Arg, -Cys, and -Pro. In some of these embodiments, the metal ion affinity peptide is chosen from His-Trp, His-Tyr, His-Gly-His, and His-Phe. In other embodiments, a metal ion affinity peptide has the formula Y-His, where Y is chosen from Gly-, Ala-, and Tyr-. See, e.g., U.S. Pat. No. 4,569,794.

In some embodiments, a metal ion affinity peptide has the formula R₁-(His-X)ₙ where R₁ is a hydrogen atom, an amino acid, or a sequence of from two amino acids to about 10 amino acids, or a sequence of from about 10 amino acids to 50 amino acids, or a polypeptide; where X is selected from Asp, Pro, Glu, Ala, Gly, Val, Ser, Leu, Ile, and Thr or a combination of any of the foregoing amino acids; and where n=3-6. See, e.g., U.S. Pat. No. 5,594,115.

In some embodiments, a metal ion affinity peptide comprises a peptide of the formula: (His-(X₁)ₙ)ₘ, wherein m≧3, wherein X₁ is any amino acid other than His, wherein n=1-3, provided that, in at least one His-(X₁)ₙ unit, n>1.

In some embodiments, a metal ion affinity peptide comprises a peptide of the formula:

(His-X₁—X₂)ₙ₁—(His-X₃—X₄—X₅)ₙ₂—(His-X₆)ₙ₃, wherein

each of X₁ and X₂ is independently an amino acid with an aliphatic or an amide side chain,

each of X₃, X₄, X₅ is independently an amino acid with a basic side chain (except His) or an acidic side chain,

each X₆ is an amino acid with an aliphatic or an amide side chain, n1 and n2 are each independently 1-3, and n3 is 1-5.

In some embodiments, each of X₁ and X₂ is independently selected from the group consisting of Leu, Ile, Val, Ala, Gly, Asn, and Gln. In other embodiments, each of X₁ and X₂ is independently selected from the group consisting of Leu, Val, Asn, and Ile. In some embodiments, each of X₃, X₄, X₅ is independently selected from the group consisting of Lys, Arg, Asp, and Glu. In some embodiments, each of X₃, X₄, X₅ is independently selected from the group consisting of Lys and Glu. In some embodiments, each X₆ is independently selected from the group consisting of Leu, Ile, Val, Ala, Gly, Asn, and Gln. In other embodiments, each X₆ is independently selected from the group consisting of Ala and Asn. In one particular embodiment, the affinity peptide has the amino acid sequence NH₂-His-Leu-Ile-His-Asn-Val-His-Lys-Glu-Glu-His-Ala-His-Ala-His-Asn-COOH (SEQ ID NO:01).

The invention further provides a metal ion affinity peptide, wherein the affinity peptide has the formula (His-Asn)ₙ, wherein n=3 to 10. In certain embodiments, n=from about 4 to about 10, and preferably from about 5 to about 10. In one particular embodiment, n=6.

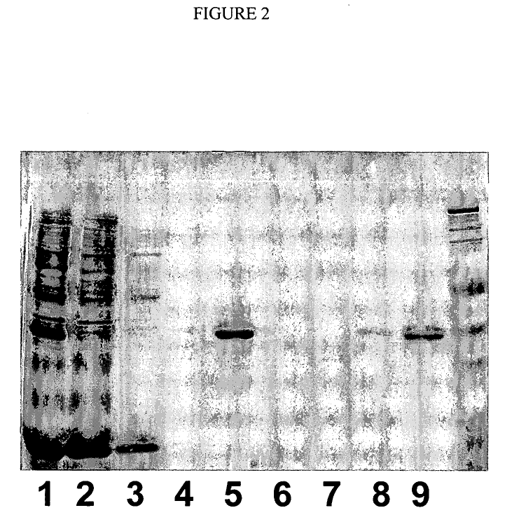

The invention further provides a metal ion affinity peptide, wherein the affinity peptide has the formula (His-X₁-X₂)ₙ, wherein each of X₁ and X₂ is an amino acid having an acidic side chain, and n=3 to 10. In one embodiment, the affinity peptide comprises the sequence (His-Asp-Asp)₆ (SEQ ID NO:02). In another embodiment, the affinity peptide comprises the sequence (His-Glu-Glu)₆ (SEQ ID NO:03). In a further embodiment, the affinity peptide comprises the sequence (His-Asp-Glu)₆ (SEQ ID NO:04). In a further embodiment, the affinity peptide comprises the sequence (His-Glu-Asp)₆ (SEQ ID NO:05). A suitable metal ion affinity peptide includes any of the metal ion affinity peptides depicted in FIGS. 2 and 3 of U.S. Published Patent Application No. 2002/0164718.

In some embodiments, metal ion affinity peptides bind to intermediate metal ions with an affinity of from about 10³ M⁻¹ to about 10⁹ M⁻¹; and to hard metal ions with an affinity of from about 10³ M⁻¹ to about 10⁹ M⁻¹.

### Fusion Proteins

As discussed above, a fusion protein that is purified according to a subject method includes a metal ion affinity peptide fused to another polypeptide, i.e., a fusion partner. A fusion protein generally comprises a polypeptide (a “fusion partner polypeptide”) fused at or near its amino- or carboxyl-terminus to a metal ion affinity peptide. The presence of the metal ion affinity peptide allows purification of the fusion protein on a metal chelating resin.

In some embodiments, a fusion protein has the formula: NH₂-ψ-ω-COOH, wherein ψ is a fusion partner polypeptide, and ω is a metal ion affinity peptide of the invention. In some of these embodiments, a fusion protein has the formula: NH₂-ψ-Z-ω-COOH, wherein Z is an intervening moiety, including but not limited to, a linker; a proteolytic cleavage site; an amino acid sequence that improves the solubility of the fusion protein; or a combination of the foregoing in any order.

In other embodiments, a fusion protein has the formula NH₂-ω-ψ-COOH. In some of these embodiments, a fusion protein has the formula: NH₂-ω-Z-ψ-COOH, wherein Z is an intervening moiety, including but not limited to, a linker; a proteolytic cleavage site; an immunological tag, or a combination of the foregoing in any order.

A linker can be any amino acid sequence that is not native to the fusion partner polypeptide, and is generally about two to about 30 amino acids in length. One non-limiting example of linker molecules is (Gly)ₙ, where n=2 to 30.

Proteolytic cleavage sites are known to those skilled in the art; a wide variety are known and have been described amply in the literature, including, e.g., Handbook of Proteolytic Enzymes (1998) A J Barrett, N D Rawlings, and J F Woessner, eds., Academic Press. Proteolytic cleavage sites include, but are not limited to, an enterokinase cleavage site: (Asp)₄Lys; a factor Xa cleavage site: Ile-Glu-Gly-Arg (SEQ ID NO:06); a thrombin cleavage site, e.g., Leu-Val-Pro-Arg-Gly-Ser (SEQ ID NO:07); a renin cleavage site, e.g., His-Pro-Phe-His-Leu-Val-Ile-His (SEQ ID NO:08); a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picomavirus (see, e.g., Sommergruber et al. (1994) Virol. 198:741-745), a Hepatitis A virus 3C cleavage site (see, e.g., Schultheiss et al. (1995) J. Virol. 69:1727-1733), human rhinovirus 2A protease cleavage site (see, e.g., Wang et al. (1997) Biochem. Biophys. Res. Comm. 235:562-566), and a picomavirus 3 protease cleavage site (see, e.g., Walker et al. (1994) Biotechnol. 12:601-605.

A fusion protein may comprise, in addition to a fusion partner polypeptide and a metal ion affinity peptide, an immunological tag. An immunological tag, if present, is present at the amino terminus, the carboxyl terminus, or disposed between the fusion partner polypeptide and the metal ion affinity peptide. Immunological tags are known in the art, and are typically a sequence of between about 6 and about 50 amino acids that comprise an epitope that is recognized by an antibody specific for the epitope. Non-limiting examples of such tags are hemagglutinin (HA; e.g., CYPYDVPDYA; SEQ ID NO:09), FLAG (e.g., DYKDDDDK; SEQ ID NO:10), c-myc (e.g., CEQKLISEEDL; SEQ ID NO:11), and the like.

A fusion protein may comprise an amino acid sequence that provides for secretion of the fusion protein from the cell. Those skilled in the art are aware of such secretion signal sequences. Secretion signals that are suitable for use in bacteria include, but are not limited to, the secretion signal of Braun's lipoprotein of E. coli, S. marcescens, E. amylosora, M. morganii, and P. mirabilis, the TraT protein of E. coli and Salmonella; the penicillinase (PenP) protein of B. licheniformis and B. cereus and S. aureus; pullulanase proteins of Klebsiella pneumoniae and Klebsiella aerogenese; E. coli lipoproteins 1pp-28, Pal, Rp1A, Rp1B, OsmB, NIpB, and Orl17; chitobiase protein of V. harseyi; the β-1,4-endoglucanase protein of Pseudomonas solanacearum, the Pal and Pcp proteins of H. influenzae; the OprI protein of P. aeruginosa; the MalX and AmiA proteins of S. pneumoniae; the 34 kda antigen and TpmA protein of Treponema pallidum; the P37 protein of Mycoplasma hyorhinis; the neutral protease of Bacillus amyloliquefaciens; and the 17 kda antigen of Rickettsia rickettsii. Secretion signal sequences suitable for use in yeast are known in the art, and can be used. See, e.g., U.S. Pat. No. 5,712,113.

Fusion partner polypeptides are of any length, e.g, from about 10 to about 5000, from about 20 to about 4500, from about 25 to about 4000, from about 50 to about 3500, from about 75 to about 3000, from about 100 to about 2500, from about 150 to about 2000, from about 200 to about 1500, from about 250 to about 1250, from about 300 to about 1000, from about 350 to about 950, from about 400 to about 900, from about 450 to about 850, from about 500 to about 800, from about 550 to about 750, or from about 600 to about 700, amino acids.

A fusion partner polypeptide can be a natural or non-natural (e.g., having an amino acid sequence not found in nature) polypeptide; a polypeptide from an animal, plant, eubacterium, archaebacterium, fungus, protozoa, or virus. A fusion partner polypeptide can be a fragment of any known naturally-occurring or non-naturally occurring polypeptide. Fragments or interest include, but are not limited to, functional domains, e.g., a catalytic domain of an enzyme, a DNA-binding domain of a transcription factor, a ligand-binding domain of a receptor, and the like; structural domains; fragments that inhibit a protein function; and the like.

The fusion partner polypeptide does not bind to the immobilized metal ion; instead, binding is mediated primarily by the metal ion affinity peptide. A fusion partner polypeptides can be any known protein, including, but not limited to, peptide hormones, enzymes, neurotransmitters, cytokines, chemokines, structural proteins, receptors, transcription factors, serum proteins, regulatory proteins, antibodies, antibiotic and bacteriostatic peptides, insecticidal, herbicidal and fungicidal peptides, and the like.

A fusion partner polypeptide can also be a protein of unknown identity or function, e.g., a protein encoded by a putative coding region identified in a sequencing project.

Suitable fusion partner polypeptides, include, but are not limited to, erythropoietin, oxytocin, vasopressin, adrenocorticotropic hormone, relaxin, epidermal growth factor, platelet-derived growth factor (PDGF), prolactin, luteinizing hormone releasing hormone (LHRH), LHRH agonists, LHRH antagonists, growth hormone (human, porcine, bovine, etc.), growth hormone releasing factor, insulin, somatostatin, glucagon, interleukin-2 (IL-2), interferon-α, β, or γ, gastrin, tetragastrin, pentagastrin, urogastrone, secretin, calcitonin, enkephalins, endorphins, angiotensins, tumor necrosis factor, nerve growth factor (NGF), granulocyte-colony stimulating factor, granulocyte macrophage-colony stimulating factor (GM-CSF), macrophage-colony stimulating factor (M-CSF), heparinase, bone morphogenic protein (BMP), atrial natriuretic peptide, glucagon-like peptide (GLP-1), interleukin-11 (IL-11), renin, bradykinin, bacitracins, polymyxins, colistins, tyrocidine, bacteriocins, gramicidins, cyclosporins, cecropins, attacins, apidaecins; polymerases, ligases, phosphorylases, kinases, phosphatases, glycosylases, sulfotransferases, lipases, dehydrogenases, reverse transcriptases; calcium channels, T-cell antigen receptor, epidermal growth factor receptor, chemokine receptors, potassium channels, serotonin receptors; tumor-associated antigens; histones, actin, myosin, tubulin, capsid proteins, group-specific antigens, viral envelope proteins; clotting factors (e.g., Factor VIII, Factor IX, etc.); etc.

### Production of Fusion Proteins

Fusion proteins are produced by any known means, including synthetic means, recombinant means, etc., which methods are well known to those skilled in the art. In some embodiments, a polynucleotide comprising a nucleotide sequence that encodes a fusion protein as described above is introduced into a cell, and the cell is cultured under conditions that favor production of the encoded fusion protein.

Polynucleotides that include a nucleotide sequence encoding a fusion protein can be prepared in a number of different ways. For example, the nucleic acid may be synthesized using solid phase synthesis techniques, as are known in the art. Oligonucleotide synthesis is also described in Edge, et al., (1981) Nature 292:756; Duckworth et al., (1981) Nucleic Acids Res 9:1691 and Beaucage, et al., (1981) Tet. Letts 22: 1859. Following preparation of the nucleic acid, the nucleic acid is then ligated to other members of the expression system to produce an expression cassette or system comprising a nucleic acid encoding the fusion protein product in operational combination with transcriptional initiation and termination regions, which provide for expression of the nucleic acid into the polypeptide products under suitable conditions.

In many embodiments, a recombinant vector (a “construct”) comprising a polynucleotide that includes a nucleotide sequence encoding a fusion protein is used. Recombinant vectors are useful for propagation of the polynucleotides (cloning vectors). They are also useful for effecting expression of a fusion protein-encoding polynucleotide in a cell (expression vectors). Some vectors accomplish both cloning and expression functions. The choice of appropriate vector is well within the skill of the art. Many such vectors are available commercially.

In some embodiments, a recombinant vector comprises a nucleotide sequence encoding a metal ion affinity peptide, and a restriction endonuclease recognition sequence for inserting a heterologous nucleic acid molecule comprising a sequence that encodes a fusion partner protein, such that when a heterologous nucleic acid molecule is inserted into the vector, the recombinant vector encodes a fusion protein as described herein. In some embodiments, more than one restriction endonuclease site is provided in a tandem and/or partially overlapping arrangement, such that a “multiple cloning site” is provided. In some embodiments, a recombinant vector further comprises control sequences, such as a promoter, for controlling transcription of a coding region for a fusion protein. Thus, in some embodiments, the recombinant vector comprises, in order from 5′ to 3′, a transcription control sequence, a restriction endonuclease recognition site, and a nucleotide sequence encoding a metal ion affinity peptide.

In other embodiments, the recombinant vector comprises, in order from 5′ to 3′, a transcription control sequence, a nucleotide sequence encoding a metal ion affinity peptide, and a restriction endonuclease recognition site. The restriction endonuclease recognition site for inserting a heterologous nucleic acid molecule is positioned relative to the sequences encoding the metal ion affinity peptide to provide for in-frame fusion of the affinity peptide with the fusion partner polypeptide, and is typically within less than about 50 bases from the sequences encoding the metal ion affinity peptide. The recombinant vector typically further comprises a nucleotide sequence encoding a selectable marker (e.g., antibiotic resistance), and an origin of replication.

A recombinant vector can further comprise a nucleotide sequence that encodes a proteolytic cleavage site, such that the fusion partner polypeptide can be cleaved away from the metal ion affinity peptide. Thus, in some embodiments, a recombinant vector comprises, in order from 5′ to 3′, a nucleotide sequence encoding a metal ion affinity peptide; a nucleotide sequence encoding a proteolytic cleavage site; and one or more restriction endonuclease recognition sites.

For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to the gene, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. After introduction of the DNA, the cells containing the construct may be selected by means of a selectable marker, the cells expanded and then used for expression.

The polypeptides may be expressed in prokaryotes or eukaryotes in accordance with conventional ways, depending upon the purpose for expression. For large-scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the protein will benefit from native folding and post-translational modifications. Small peptides can also be synthesized in the laboratory. Polypeptides that are subsets of the complete amino acid sequence may be used to identify and investigate parts of the protein important for function, or to raise antibodies directed against these regions.

A variety of host-vector systems may be utilized to propagate and/or express the polynucleotide. Such host-vector systems represent vehicles by which coding sequences of interest may be produced and subsequently purified, and also represent cells that may, when transformed or transfected with the appropriate nucleotide coding sequences, produce fusion polypeptides of the invention. These include, but are not limited to, microorganisms (e.g., E. coli, B. subtilis) transformed with recombinant bacteriophage vectors, plasmid DNA, or cosmid DNA vectors comprising the fusion protein-encoding polynucleotides; yeast (e.g., Saccharomyces, Pichia) transformed with recombinant yeast vectors comprising fusion protein-encoding polynucleotides); insect cell systems (e.g., Spodoptera frugiperda) infected with recombinant virus expression vectors (e.g., baculovirus vectors, many of which are commercially available, including, for example, pBacPAK8, and BacPAK6) comprising fusion protein-encoding polynucleotides; plant cell systems; or mammalian cell systems (e.g., COS, CHO, BHK, 293, 3T3) harboring recombinant vectors comprising mammalian promoters (e.g., metallothionein promoter) or promoters from viruses which replicate in mammalian cells (e.g., adenovirus late promoter; vaccinia virus promoter, and the like).

Examples of prokaryotic cloning vectors which find use in propagating fusion protein-encoding polynucleotides are pBR322, M13 vectors, pUC18, pcDNA, and pUC19. Prokaryotic expression vectors which find use in expressing subject polypeptides in prokaryotic cells include pTrc99A, pK223-3, pEZZ18, pRIT2T, and pMC1871.

Eukaryotic expression vectors which find use in expressing fusion protein-encoding polynucleotides and fusion polypeptides in eukaryotic cells include commercially available vectors such as pSVK3, pSVL, pMSG, pCH110, pMAMneo, pMAMneo-LUC, pPUR, and the like.

Generally, a bacterial host will be transformed to contain the expression system using a vector. A variety of vectors may be employed so long as they introduce the expression system into the host in a manner whereby the product encoded by the expression system can be expressed.

Generally, the expression cassette will be a plasmid that provides for expression of the encoded fusion polypeptide under appropriate conditions, i.e. in a host cell. The expression vector will typically comprise a replicon, which includes the origin of replication and its associated cis-acting control elements. Representative replicons that may be present on the expression vector include: pMB1, p15A, pSC101 and ColE1. Expression vectors generally have convenient restriction sites located near the promoter sequence to provide for the insertion of nucleic acid sequences encoding heterologous proteins.

In addition, the expression vector will also typically comprise a marker which provides for detection of the clones that have been transformed with the vector. A variety of markers are known and may be present on the vector, where such markers include those that confer antibiotic resistance, e.g. resistance to ampicillin, tetracycline, chloramphenicol, kanamycin (neomycin), markers that provide for histochemical detection, etc. Specific vectors that may find use in the subject methods include: pBR322, pUC18, pUC19, pcDNA, and the like. Introduction of the nucleic acid encoding the fusion protein product into the expression vector is accomplished by cutting the expression vector and inserting the polynucleotide encoding the desired product.

Following preparation of the expression vector comprising the nucleic acid, the expression vector will be introduced into an appropriate host cell for production of the fusion polypeptide, i.e. a host cell will be transformed with the expression vector. Transformation of host cells may be accomplished in any convenient manner, where two representative means of transformation are treatment with divalent cation transformation compositions and electrotransformation. In transformation through divalent cation treatment, the host cells are typically incubated with the one or more divalent cations, e.g. CaCl₂, which serves to make the host cell permeable to the vector DNA. See Cohen et al. (1972) Proc. Nat'l. Acad. Sci. U.S.A. 69:2110. Other agents with which the host cells may also be incubated include DMSO, reducing agents, hexaminecobalt and the like, where such agents serve to improve the efficiency of transformation. In electrotransformation (also known as transformation by electroporation) host cells are subject to an electrical pulse in the presence of the vector in a manner sufficient for the vector to enter the host cells. See Dower et al. (1988) Nucleic Acids Research 16:6127.

A variety of host cells are suitable and may be used in the production of the fusion polypeptides. Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilbum et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

Plant cells. Plant cell culture is amply described in various publications, including, e.g., Plant Cell Culture: A Practical Approach, (1995) R. A. Dixon and R. A. Gonzales, eds., IRL Press; and U.S. Pat. No. 6,069,009.

### UTILITY

The subject methods find use in a number of different applications where protein purification is desired.

Metal ion affinity peptide-tagged recombinant proteins are useful for the study of protein-protein interactions and nucleic acid molecule-protein interactions, using solid phase IMAC having bound thereto a fusion protein. In these applications, the solid phase IMAC serves to anchor a fusion protein, thereby immobilizing the fusion protein. Analysis of protein-protein interactions and nucleic acid molecule-protein interactions are generally carried out by detecting a protein or nucleic acid molecule bound to the fusion partner polypeptide of an immobilized fusion protein. Detection can be carried out using any known method, and in many instances involves use of a detectably labeled reagent, e.g., a detectably labeled antibody specific for a given protein, a detectably labeled nucleic acid molecule that hybridizes to a nucleic acid molecule to be detected, and the like.

Also contemplated is the use of the subject methods in high throughput systems, e.g., where protein purification of a large number of samples is desired. High throughput systems find use, e.g., in massive parallel gene expression experiments, e.g., to determine the effect of an agent on synthesis of a protein or set of proteins; to analyze developmental stage-specific, or tissue-specific synthesis of a protein; to analyze the phosphorylation state of a protein; and the like.

The methods are useful in applications to characterize a protein of unknown identity or function. For example, a putative coding region identified in a sequencing project is cloned into an expression vector such that the encoded protein comprises a metal ion affinity peptide, the vector is introduced into a host cell for transcription and translation of the putative coding region, and the protein purified, as described in more detail below. The function of the protein can then be determined, using any known assay method, including, but not limited to, assays for protein-protein interaction; assays for protein-nucleic acid molecule interactions; assays for enzymatic activity; and the like.

The methods are further useful in carrying out enzymatic reactions. A fusion protein having as a fusion partner a protein with enzymatic activity is immobilized on an IMAC solid support, and contacting the immobilized enzyme with a substrate under conditions appropriate to the enzymatic activity of the enzyme. In general, the immobilized enzyme is purified using a method as described herein before contacting the enzyme with a substrate. The product(s) of the enzymatic reaction, which are in the medium (e.g., the buffer in which the enzymatic reaction took place), are readily collected by separating the medium from the IMAC solid support. Separation of the medium from the IMAC solid support is achieved using standard methods, e.g., using standard techniques of column chromatography, centrifugation, and the like.

### KITS

The invention provides kits for practicing the subject methods. Thus, the invention provides a kit for purification of a fusion protein comprising a metal ion affinity peptide.

In general, a subject kit for purifying a protein comprises at least a first metal ion chelate resin comprising a first immobilized metal ion and a second metal ion affinity resin comprising a second immobilized metal ion. In some embodiments, the first metal ion is a hard metal ion (e.g., Fe³⁺, Ca²⁺ or Al³⁺), and the second metal ion is an intermediate metal ion (e.g., Cu²⁺, Ni²⁺, Zn²⁺ or Co²⁺). In other embodiments, the first metal ion is an intermediate metal ion; and the second metal ion is a hard metal ion. In some embodiments, the kit further includes one or more of: an extraction buffer; an equilibration buffer; a wash buffer; and an elution buffer. In some embodiments, the kit further includes one or more columns. For example, in some embodiments, a subject kit includes a first metal ion chelate resin comprising a first immobilized metal ion, where the first immobilized metal ion is an intermediate metal ion; a second metal ion chelate resin comprising a second immobilized metal ion, where the second immobilized metal ion is a hard metal ion; at least one wash buffer (e.g., one or more wash buffers for the first metal ion chelate resin; and one or more wash buffers for the second metal ion chelate resin); an equilibration buffer; an elution buffer; and at least one column (e.g., one column for the first metal ion chelate resin; and a second column for the second metal ion chelate resin).

In some embodiments, a subject kit comprises a metal ion affinity resin, an extraction buffer, an equilibration buffer, a wash buffer, and an elution buffer. In some of these embodiments, a kit further comprises a column for use in protein purification. In other embodiments, the metal ion affinity resin is provided attached to a solid support.

In some embodiments, a kit of the invention comprises a recombinant vector that includes a nucleotide sequence encoding a fusion protein that includes a metal ion affinity peptide. In some embodiments, a kit further comprises an appropriate restriction enzyme(s), ligases, and other reagents for inserting a heterologous nucleic acid molecule into the recombinant vector. The kit may further comprise bacteria; reagents for introducing the recombinant vector into the bacteria; reagents for selecting bacteria that comprise the recombinant vector; reagents for inducing expression of the fusion protein; and reagents for disrupting bacteria after a fusion protein is produced.

In other embodiments, a kit comprises, in addition to a recombinant vector, and optionally other components as described above, one or more metal ion affinity resins. In some of these embodiments, a kit further comprises, extraction, wash, and elution buffers, and, in some embodiments, further comprises one or more columns.

The kit may optionally provide additional components that are useful in the procedure, including, but not limited to, buffers, developing reagents, labels, reacting surfaces, means for detections, control samples, standards, instructions, and interpretive information.

Finally, in many embodiments of the subject kits, the kits will further include instructions for practicing the subject methods or means for obtaining the same (e.g., a website URL directing the user to a webpage which provides the instructions), where these instructions are typically printed on a substrate, which substrate may be one or more of: a package insert, the packaging, reagent containers and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Bi-MAC (Bi-Metal Affinity Chromotography)

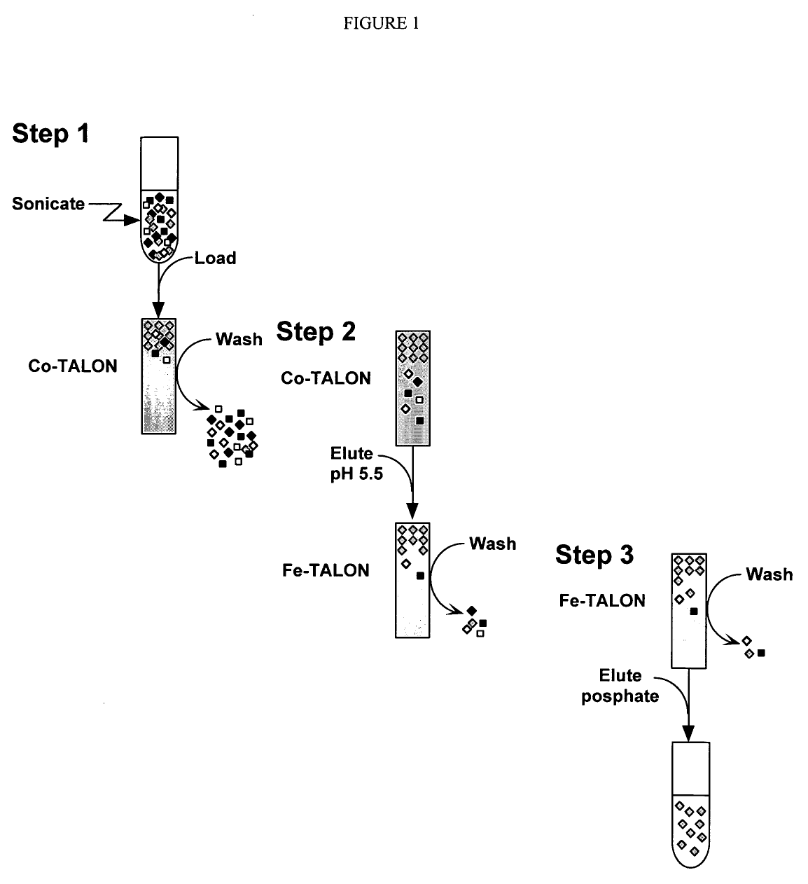

An illustrative purification protocol for Bi-MAC is shown in FIG. 1. This protocol was carried out for HAT-GFPuv, and HAT-DHFR, which were generated as described in U.S. Patent Publication No. 2002/0164718, the contents of which are incorporated herein by reference. The 3-step purification procedure was carried out as follows. A sonicate containing a fusion protein (e.g., HAT-GFPuv or HAT-DHFR) having a metal ion affinity peptide was loaded on a first column. The first column contained a first metal ion chelate resin having a Co²⁺ ion (Co-TALON™). The first column was washed (first with equilibration buffer, then with 5 mM imidazole), then eluted with a buffer (pH 5.5). The eluate was applied to a second column. The second column contained a second metal ion chelate resin having an Fe³⁺ ion (Fe-TALON™). The second column was washed (first with a pH 5.5 wash solution, then with a pH 7.3 wash solution), then eluted with phosphate.

FIG. 2 shows the results of an analysis of samples taken at various steps during purification. Lane 1: starting sample; Lane 2: non-adsorbed material from Co-TALON column (first column); Lane 3: equilibration buffer wash of Co-TALON column; Lane 4: 5 mM imidazole wash of Co-TALON column; Lane 5: eluate from Co-TALON, eluted with pH 5.5 buffer; Lane 6: non-adsorbed material from Fe-TALON column; Lane 7: pH 5.5 wash of Fe-TALON column; Lane 8: pH 7.3 wash of Fe-TALON column; Lane 9: eluate from Fe-TALON column; Lane 10: molecular weight markers.

The results of the protein analysis of samples taken at various steps during purification is shown in Table 1, below.

TABLE 1

| | Volume, | | | |
|---|---|---|---|---|
| Fraction | mL | Protein, mg/mL | Total, mg | Ratio, % |
| OS | 7 | 0.88 | 6.20 | 100 |
| NA-Co | 6 | 0.65 | 3.90 | 63 |
| W1 | 9 | 0.09 | 0.80 | 14 |
| W2 | 8 | 0.01 | 0.08 | 2 |
| Eluate-Co | 4 | 0.34 | 1.37 | 22 |
| NA-Fe | 4 | 0 | | |
| W3 | 7 | 0.06 | 0.40 | 6 |
| Eluate-Fe | 4 | 0.15 | 0.59 | 10 |

OS, original sample as applied to Co-TALON column; NA-Co, non-adsorbed material from Co-TALON column; W1, wash 1 of Co-TALON column; W2, wash 2 of Co-TALON column; Eluate-Co, eluate from Co-TALON column; NA-Fe, non-adsorbed material from Fe-TALON column; W3, wash of Fe-TALON column; Eluate-Fe, eluate from Fe-TALON column.

It is evident from the above results and discussion that the invention provides metal ion affinity peptides, fusion proteins thereof, and methods of purifying same, which provide for improved purification of proteins.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of purifying a fusion protein, the method comprising:
(a) contacting a sample comprising a fusion protein having a metal ion affinity peptide with a first metal ion chelate resin comprising an immobilized first metal ion;
(b) eluting any resultant bound fusion protein from said resin to produce a first eluate;
(c) contacting the first eluate with a second metal ion affinity resin comprising a second immobilized metal ion; and
(d) eluting any resultant bound fusion protein from said first and second resins to produce a product eluate comprising a purified fusion protein.

2. The method according to claim 1, wherein the first metal ion is a hard metal ion, and the second metal ion is an intermediate metal ion.

3. The method according to claim 2, wherein the hard metal ion is chosen from Fe³⁺, Ca²⁺ and Al³⁺; and the intermediate metal ion is chosen from Cu²⁺, Ni²⁺, Zn²⁺ and Co²⁺.

4. The method according to claim 1, wherein the first metal ion is an intermediate metal ion, and the second metal ion is a hard metal ion.

5. The method according to claim 4, wherein the hard metal ion is chosen from Fe³⁺, Ca²⁺ and Al³⁺; and the intermediate metal ion is chosen from Cu²⁺, Ni²⁺, Zn²⁺ and Co²⁺.

6. The method according to claim 1, wherein the first metal ion is Co²⁺, and the second metal ion is Fe³⁺.

7. The method according to claim 1, wherein the first metal ion is Fe³⁺, and the second metal ion is Co²⁺.

8. The method according to claim 1, further comprising a wash step between steps (a) and (b) and between steps (c) and (d).

9. The method according to claim 1, wherein the contacting and eluting steps are carried out under native conditions.

10. The method according to claim 1, wherein the contacting and eluting steps are carried out under denaturing conditions.

11. A kit for purifying a protein, said kit comprising at least a first metal ion chelate resin comprising a first immobilized metal ion and a second metal ion affinity resin comprising a second immobilized metal ion.

12. The kit according to claim 11, wherein the first metal ion is a hard metal ion, and the second metal ion is an intermediate metal ion.

13. The kit according to claim 12, wherein the hard metal ion is chosen from Fe³⁺, Ca²⁺ and Al³⁺; and the intermediate metal ion is chosen from Cu²⁺, Ni²⁺, Zn²⁺ and Co²⁺.

14. The kit according to claim 11, wherein the first metal ion is an intermediate metal ion, and the second metal ion is a hard metal ion.

15. The kit according to claim 14, wherein the hard metal ion is chosen from Fe³⁺, Ca²⁺ and Al³⁺; and the intermediate metal ion is chosen from Cu²⁺, Ni²⁺, Zn²⁺ and Co²⁺.

16. The kit according to claim 11, further comprising:
an extraction buffer;
a wash buffer; and
an elution buffer.

17. The kit according to claim 11, further comprising a column.
